(19) United States
(12) Patent Application Publication
(10) Pub. No.: US 2024/0374860 A1
(43) Pub. Date: Nov. 14, 2024

(54) MOTION SICKNESS REDUCTION SYSTEM AND METHOD

(71) Applicant: HYUNDAI MOBIS CO., LTD., Seoul (KR)

(72) Inventors: Ki Hee PARK, Gwacheon-si (KR); Yeong Hun PARK, Seoul (KR); Chang Won LEE, Seoul (KR)

(73) Assignee: HYUNDAI MOBIS CO., LTD., Seoul (KR)

(21) Appl. No.: 18/521,564

(22) Filed: Nov. 28, 2023

(30) Foreign Application Priority Data
May 10, 2023 (KR) 10-2023-0060366
May 10, 2023 (KR) 10-2023-0060367

(51) Int. Cl. A61M 21/00; B60R 16/037

(52) U.S. Cl. CPC A61M 21/00; B60R 16/037; A61M 2021/0027; A61M 2021/005

(57) ABSTRACT

A motion sickness reduction system of a passenger of a vehicle includes a state information acquirer to collect state information of the passenger, a behavior information acquirer to collect real-time driving information of the vehicle from a sensor located in the vehicle, a processor to determine a motion sickness state of the passenger based on the collected state information, and generate motion sickness reduction information based on the determined motion sickness state; and an output unit to output the generated motion sickness reduction information. The generated motion sickness reduction information output by the output unit is at least one of visually recognizable by the passenger, auditorily recognizable by the passenger, or a combination thereof.

### CROSS-REFERENCE TO RELATED APPLICATION(S)

This application claims the benefit under 35 U.S.C. § 119 (a) of Korean Patent Application No. 10-2023-0060366, filed on May 10, 2023, and Korean Patent Application No. 10-2023-0060367, filed on May 10, 2023, in the Korean Intellectual Property Office, the entire disclosures of which are incorporated by reference for all purposes.

### BACKGROUND

### 1. Field

The present disclosure relates to a motion sickness reduction system and method for reducing motion sickness of a vehicle passenger by providing information based on which the vehicle passenger visually or audibly recognizes real-time driving information of a vehicle.

### 2. Description of Related Art

Motion sickness may occur when a person is exposed to one or more certain motions for a long time. At this time, factors such as temperature, smell, emotions, and digestive power may promote motion sickness.

In particular, many people experience carsickness, and many solutions are proposed to suppress this. Suspensions taken before boarding a vehicle are representative, but these suspensions contain ingredients such as scopolamine, dimenhydrinate, diphenhydramine, promethazine, and meclizine, which have many side effects.

Accordingly, anti-motion sickness medications in the form of patches have been mainly used recently. However, patch-type anti-motion sickness medications do not have the same effect on everyone and in all situations, and thus consumers are dissatisfied with their effectiveness.

Motion sickness, which is accompanied by dizziness and vomiting when riding a vehicle, causes temporary confusion in the brain when there is a mismatch in input between the sensory organs (vision, somatosensory, semicircular canal, and the like) that maintain balance or detect movement and posture.

People remember in their minds the responses of sensory organs such as eyes and ears to muscle movements, and when similar movements occur later, the sensory organs use memorized information to predict in advance, prepare, and respond. However, when riding in a vehicle, there is no muscle movement due to movement or movement is different from existing memories, and thus there is a mismatch in sensation and motion sickness occurs.

According to the related art, a technology for detecting motion sickness uses only extremely limited vibration information of a vehicle, and thus it is not possible to accurately detect motion sickness of passengers.

Therefore, to reduce motion sickness in a vehicle passenger, an element is needed to reduce motion sickness in the vehicle passenger by reducing a difference between a motion actually felt by the passenger in relation to real-time driving information of a vehicle and the motion perceived by the brain of the passenger.

### SUMMARY

This Summary is provided to introduce a selection of concepts in a simplified format that are further described below in the Detailed Description. This Summary is not intended to identify key features or essential features of the claimed subject matter, nor is it intended to be used as an aid in determining the scope of the claimed subject matter.

In a general aspect of the disclosure, a motion sickness reduction system of a passenger of a vehicle, the motion sickness reduction system includes: a state information acquirer to collect state information of the passenger; a behavior information acquirer to collect real-time driving information of the vehicle from a sensor located in the vehicle; a processor to determine a motion sickness state of the passenger based on the collected state information, and generate motion sickness reduction information based on the determined motion sickness state; and an output unit to output the generated motion sickness reduction information, wherein the generated motion sickness reduction information output by the output unit is at least one of visually recognizable by the passenger, auditorily recognizable by the passenger, or a combination thereof.

The output unit may output information through at least one of a display, a front window located in the vehicle, or a combination thereof.

The motion sickness reduction information generated by the processor may include an augmented reality video reflecting the real-time driving information of the vehicle, and the generated augmented reality video may output to at least one of the display, a lower end of the front window, or a combination thereof.

The display may include: a front display located on a front side of the vehicle; and a rear seat entertainment (RSE) display located on a roof of the vehicle.

The motion sickness reduction system may further include a driving module located on the roof of the vehicle, wherein the driving module may rotate or position the RSE display.

The output unit may output information through at least one of a side window, a sunroof located in the vehicle, or a combination thereof.

The motion sickness reduction information generated by the processor may include a gradation change reflecting the real-time driving information of the vehicle through at least one of a polymer-dispersed liquid crystal (PDLC) film located in the side window, the sunroof, or a combination thereof.

The generated motion sickness reduction information that is recognized by the passenger may be comparable with actual motion sickness felt by the passenger.

The output unit may output information through a speaker unit of a seat headrest located in the vehicle, and the speaker unit may include a first speaker located at one side of the seat headrest and a second speaker located at another side of the seat headrest.

The motion sickness reduction information generated by the processor may include a spatial acoustic sound that reflects the real-time driving information of the vehicle, and the generated spatial acoustic sound may be sequentially output from the first speaker to the second speaker depending on a rotation direction of the vehicle.

The processor may generate the spatial acoustic sounds with different tones, intervals, and cycles based on the real-time driving information of the vehicle.

The motion sickness reduction system may further include a user input unit to receive output of the passenger, wherein the output unit may output the spatial acoustic sound based on the received output of the passenger.

The output unit may output a spatial acoustic sound reflecting the real-time driving information of the vehicle through an acoustic device of the vehicle.

In another general aspect of the disclosure, a method of reducing motion sickness of a passenger of a vehicle includes: collecting state information of the passenger; collecting real-time driving information of the vehicle from a sensor located in the vehicle; determining a motion sickness state of the passenger based on the collected state information; generating motion sickness reduction information based on the determined motion sickness state; and outputting the generated motion sickness reduction information, wherein the generated motion sickness reduction information that is output is at least one of visually recognizable by the passenger, auditorily recognizable by the passenger, or a combination thereof.

The generating of the motion sickness reduction information may include generating an augmented reality video reflecting the real-time driving information of the vehicle, and the outputting of the generated motion sickness reduction information may include outputting the generated augmented reality video on at least one of a display, a lower end of a front window located in the vehicle, or a combination thereof.

The generating of the motion sickness reduction information may include changing a gradation reflecting the real-time driving information of the vehicle through a polymer-dispersed liquid crystal (PDLC) film located in at least one of a side window, a sunroof of the vehicle, or a combination thereof, and the outputting of the generated motion sickness reduction information may include outputting of the generated gradation change on at least one of the side window, the sunroof, or a combination thereof.

The generated motion sickness reduction information that is recognized by the passenger may be comparable with actual motion sickness felt by the passenger.

The generating of the motion sickness reduction information may include generating a spatial acoustic sound that reflects the real-time driving information of the vehicle, and the outputting of the generated motion sickness reduction information may include outputting information from a first speaker to a second speaker depending on a rotation direction of the vehicle.

The generating of the spatial acoustic sound may include generating the spatial acoustic sounds with different tones, intervals, and cycles based on the real-time driving information of the vehicle.

The outputting of the generated motion sickness reduction information may include outputting a spatial acoustic sound that reflects the real-time driving information of the vehicle through an acoustic device of the passenger connected to the vehicle.

### BRIEF DESCRIPTION OF THE DRAWINGS

FIG. 1 is a block diagram of a motion sickness reduction system according to an embodiment of the present disclosure.

FIG. 2 is a diagram for explaining an output unit of FIG. 1 in more detail.

FIG. 3 is a diagram showing an augmented reality video reflecting real-time driving information of a vehicle output through a front window in a motion sickness reduction system according to an embodiment of the present disclosure.

FIG. 4 is a diagram showing a rear seat entertainment (RSE) display of a motion sickness reduction system according to an embodiment of the present disclosure.

FIG. 5 is a diagram showing a gradation change reflecting real-time driving information of a vehicle output through a polymer-dispersed liquid crystal (PDLC) film in a motion sickness reduction system according to an embodiment of the present disclosure.

FIG. 6 is a diagram for explaining a method of reducing motion sickness according to an embodiment of the present disclosure.

FIG. 7 is a diagram for explaining an output unit of FIG. 1 in more detail.

FIG. 8 is a diagram for explaining a spatial acoustic sound that reflects real-time driving information of a vehicle output through a seat headrest in a motion sickness reduction system according to an embodiment of the present disclosure.

FIG. 9 is a diagram for explaining a method of reducing motion sickness according to an embodiment of the present disclosure.

### DETAILED DESCRIPTION

Description will now be given in detail according to exemplary embodiments disclosed herein, with reference to the accompanying drawings. The same or equivalent components may be provided with the same reference numbers, and the description thereof will not be repeated. As used herein, the suffixes “module” and “part” are added or used interchangeably to facilitate preparation of this specification and are not intended to suggest distinct meanings or functions. In describing embodiments disclosed in this specification, relevant well-known technologies may not be described in detail in order not to obscure the subject matter of the embodiments disclosed in this specification. In addition, it should be noted that the accompanying drawings are only for easy understanding of the embodiments disclosed in the present specification, and should not be construed as limiting the technical spirit disclosed in the present specification. As such, the present disclosure should be construed to extend to any alterations, equivalents and substitutes in addition to those which are particularly set out in the accompanying drawings.

Although the terms first, second, etc. may be used herein to describe various elements, these elements should not be limited by these terms. These terms are generally only used to distinguish one element from another.

It will be understood that when an element is referred to as being “connected with” another element, the element can be directly connected with the other element or intervening elements may also be present. In contrast, it will be understood that when an element is referred to as being “directly connected with” another element, there are no intervening elements present.

A singular representation may include a plural representation unless it represents a definitely different meaning from the context.

The terms such as “include” or “have” used herein are intended to indicate that features, numbers, steps, operations, elements, components, or combinations thereof used in the following description exist and it should be thus understood that the possibility of existence or addition of one or more different features, numbers, steps, operations, elements, components, or combinations thereof is not excluded.

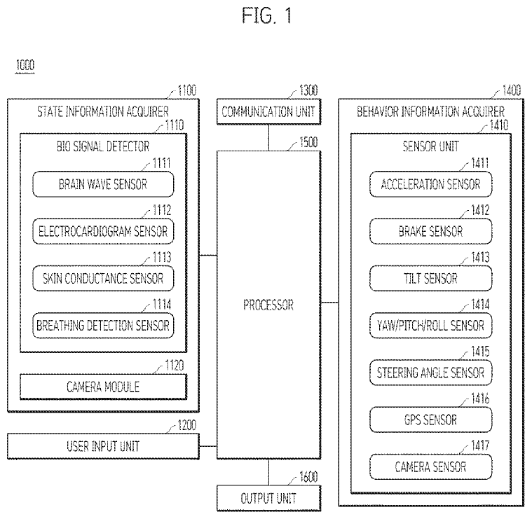

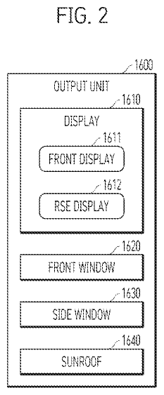

FIG. 1 is a block diagram of a motion sickness reduction system 1000 according to an embodiment of the present disclosure. FIG. 2 is a diagram for explaining an output unit 1600 of FIG. 1 in more detail.

The motion sickness reduction system 1000 according to an embodiment of the present disclosure may include a state information acquirer 1100, a user input unit 1200, a communication unit 1300, a behavior information acquirer 1400, a processor 1500, and the output unit 1600.

First, the state information acquirer 1100 may include a bio signal detector 1110 and a camera module 1120. Here, the bio signal detector 1110 may include a brain wave sensor 1111, an electrocardiogram sensor 1112, a skin conductance sensor 1113, and a breathing detection sensor 1114. Through the bio signal detector 1110 described above, a brain wave signal, an electrocardiogram signal, a skin conductance signal, or a breathing signal of a passenger may be measured, and through the camera module 1120, an image of the passenger may be monitored, and state information of the passenger may be collected.

That is, the motion sickness reduction system 1000 according to an embodiment of the present disclosure may collect state information of the passenger through the state information acquirer 1100 and determine a motion sickness state of the passenger based on the collected state information.

The user input unit 1200 may receive input from the passenger. For example, the passenger may select whether to output motion sickness reduction information, which will be described later, through the output unit 1600. In other words, the passenger may determine whether to output motion sickness reduction information depending on a normal state, an uncomfortable state of riding a vehicle, or a motion sickness state.

The communication unit 1300 may transmit the bio signal of the passenger, measured through the bio signal detector 1110 described above, to the processor 1500 wirelessly using communications such as Bluetooth, infrared communication, RFID, and UWB, or may transmit the bio signal to the passenger by wire to the processor 1500. Additionally, the communication unit 1300 may be connected wirelessly or wired to various devices carried by the passenger.

The behavior information acquirer 1400 may collect real-time driving information of a vehicle from a sensor located in the vehicle. Here, a sensor unit 1410 may include an acceleration sensor 1411, a brake sensor 1412, a tilt sensor 1413, a yaw/pitch/roll sensor 1414, a steering angle sensor 1415, a global positioning system (GPS) sensor 1416, and a camera sensor 1417. That is, the motion sickness reduction system 1000 according to an embodiment of the present disclosure may collect real-time driving information of the vehicle, such as straight driving, turning driving, change in speed, acceleration, and change in vehicle height, through the sensor unit 1410 described above.

As described above, motion sickness, which is accompanied by dizziness and vomiting when riding a vehicle, causes temporary confusion in the brain when there is a mismatch in input between the sensory organs (vision, somatosensory, semicircular canal, and the like) that maintain balance or detect motion and posture and in this regard, people remember in their minds responses of the sensory organs such as eyes and ears to muscle motions, and when similar motions occur later, the sensory organs use the memorized information to predict in advance, prepare, and respond.

However, in the vehicle, there is no motion of the muscles due to the motion, or the motion is different from the existing memory. Here, according to the related art, a to detect motion sickness, only extremely limited vibration information of a vehicle is used, and thus it is not possible to accurately detect motion sickness of passengers.

An object of the motion sickness reduction system 1000 according to an embodiment of the present disclosure is to reduce motion sickness of a vehicle passenger by reducing a difference between a motion actually felt by the passenger in relation to real-time driving information of the vehicle and a motion perceived by the brain of the passenger through the state information acquirer 1100 for collecting state information of state information of the passenger and the behavior information acquirer 1400 for collecting real-time driving information of the vehicle.

The processor 1500 may determine a motion sickness state of the passenger based on the state information collected through the state information acquirer 1100, and create motion sickness reduction information based on which the passenger visually recognizes the real-time driving information of the vehicle based on the determined motion sickness state.

The output unit 1600 may output the motion sickness reduction information created through the processor 1500. In more detail, referring to FIG. 2, the output unit 1600 may include a display unit 1610, a front window 1620, a side window 1630, and a sunroof 1640. Here, the display unit 1610 may include a front display 1611 located on a front side of the vehicle and a rear seat entertainment (RSE) display 1612 located on a roof of the vehicle.

The motion sickness reduction information created by the processor 1500 and the motion sickness reduction information output through the output unit 1600 will be described below.

The block diagram of the motion sickness reduction system 1000 shown in FIGS. 1 and 2 is only a block diagram for an embodiment of the present disclosure, and each component of the block diagram may be integrated, added, or omitted depending on the specifications of the motion sickness reduction system 1000 actually implemented. That is, as needed, two or more components may be combined into one component, or one component may be divided into two or more components. In addition, a function performed in each block is intended to explain an embodiment of the present disclosure, and a specific operation or device thereof does not propose the scope of the present disclosure.

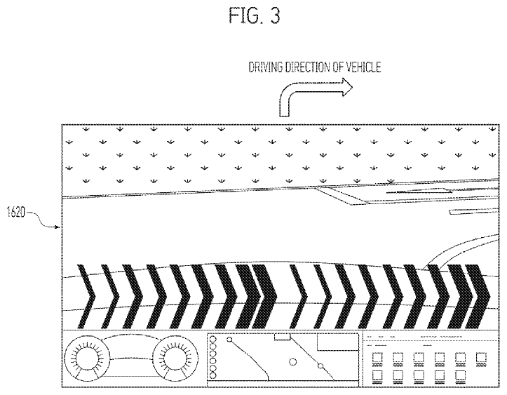

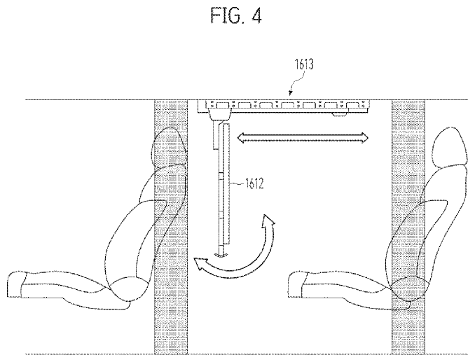

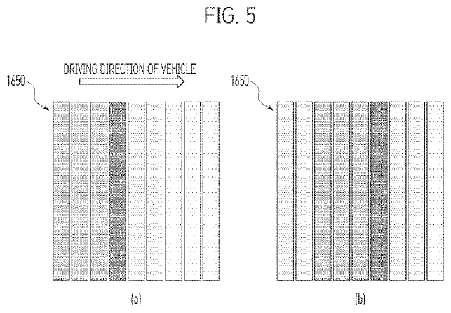

FIG. 3 is a diagram showing an augmented reality video reflecting real-time driving information of a vehicle output through the front window 1620 in the motion sickness reduction system 1000 according to an embodiment of the present disclosure. FIG. 4 is a diagram showing the RSE display 1612 of the motion sickness reduction system 1000 according to an embodiment of the present disclosure. FIG. 5 is a diagram showing a gradation change reflecting real-time driving information of a vehicle output through a polymer-dispersed liquid crystal (PDLC) film 1650 in the motion sickness reduction system 1000 according to an embodiment of the present disclosure.

Referring to FIGS. 1 and 2, in the motion sickness reduction system 1000 according to an embodiment of the present disclosure, the output unit 1600 may output the motion sickness reduction information created by the processor 1500 through the display unit 1610 or the front window 1620. Here, the motion sickness reduction information created by the processor 1500 may include augmented reality video reflecting real-time driving information of the vehicle.

FIG. 3 is a diagram showing an augmented reality video reflecting real-time driving information of a vehicle output through the front window 1620 in the motion sickness reduction system 1000 according to an embodiment of the present disclosure, and as shown in FIG. 3, when a driving direction of the vehicle is right, a right arrow indicating a rotation direction may be continuously displayed on the front window 1620.

In particular, to minimize an effect of an actual driving environment visible to the passenger, the augmented reality video may be output through a lower end of the front window 1620, that is, a bonnet portion of the vehicle of a field of vision through the front window 1620 of the passenger.

In addition, the created augmented reality video may vary in the form of output according to a speed, acceleration, and driving direction of the vehicle by applying the real-time driving information of the vehicle. For example, depending on the speed of the vehicle, an interval at which the output augmented reality video is displayed may vary or the augmented reality video may also output with different thicknesses.

FIG. 4 is a diagram showing the RSE display 1612 in the motion sickness reduction system 1000 according to an embodiment of the present disclosure, and to reduce motion sickness of a passenger riding in a rear row of a vehicle, motion sickness reduction information created by the processor 1500 described above with reference to FIG. 1.

Here, the RSE display 1612 may be located in a roof of the vehicle, and a driving module 1613 that rotates or positions the RSE display 1612 may be located on the roof of the vehicle. Here, the driving module 1613 may include, for example, a rotating motor rotating the RSE display 1612, and may also include a ball screen moving the RSE display 1612 in a forward and backward direction.

FIG. 5 is a diagram showing a gradation change reflecting real-time driving information of a vehicle output through the PDLC film 1650 in the motion sickness reduction system 1000 according to an embodiment of the present disclosure.

Referring to FIGS. 1 and 2, in the motion sickness reduction system 1000 according to an embodiment of the present disclosure, the output unit 1600 may output the motion sickness reduction information created by the processor 1500 through the side window 1630 or the sunroof 1640 located in the vehicle. Here, the motion sickness reduction information created by the processor 1500 may include a gradation change reflecting the real-time driving information of the vehicle through the PDLC film 1650 located in the side window 1630 or the sunroof 1640.

Here, the PDLC film 1650 is a thin film having a thickness of 10 to 30 micrometers according to an embodiment of the present disclosure, an electrode supplying power may be provided on a side surface of the PDLC film 1650, and the aforementioned gradation change may be output to the side window 1630 or the sunroof 1640 through the output unit 1600 by supplying the power to the electrode through the processor 1500 described above with reference to FIG. 1.

That is, as shown in FIG. 5, the motion sickness reduction system 1000 according to an embodiment of the present disclosure may output the gradation change to the side window 1630 or the sunroof 1640 through the PDLC film 1650 to allow the passenger to visually recognize the real-time driving information of the vehicle, thereby reducing a difference between a motion that the passenger actually feels with respect to the real-time driving information of the vehicle and a motion recognized by the brain of the passenger.

According to an embodiment of the present disclosure, when the gradation change is output to the sunroof 1640 of the vehicle according to a seat position of the vehicle or a seating position of the passenger, a gradation change in a curved form depending on a rotation direction of the vehicle may be output. The gradient change may be output in consideration of a radius of the curved form depending on a rotation angle of the vehicle. The form of gradation changes may vary depending on the speed, acceleration, and driving direction of the vehicle, reflecting the real-time driving information of the vehicle.

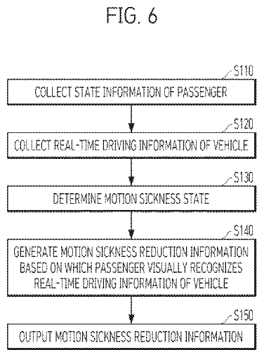

FIG. 6 is a diagram for explaining a method of reducing motion sickness according to an embodiment of the present disclosure.

First, the state information of the passenger may be collected (S110) and the real-time driving information of the vehicle may be collected from a sensor located in the vehicle (S120). Then, the motion sickness of the passenger may be determined based on the collected state information (S130), and the motion sickness reduction information based on which the passenger visually recognizes the real-time driving information of the vehicle based on the determined motion sickness state may be created (S140). The created motion sickness reduction information may be output (S150) to reduce motion sickness of the vehicle passenger.

In more detail, as described above with reference to FIGS. 1 to 5, the creating of the motion sickness reduction information in the method of reducing motion sickness according to an embodiment of the present disclosure may include creating an augmented reality video reflecting the real-time driving information of the vehicle. The outputting of the created motion sickness reduction information may include outputting of the motion sickness reduction information created on the display unit 1610 or the lower end of the front window 1620 located in the vehicle.

In addition, the creating of the motion sickness reduction information in the method of reducing motion sickness according to an embodiment of the present disclosure may include creating a gradation change reflecting the real-time driving information of the vehicle through the PDLC film 1650 located in the side window 1630 or the sunroof 1640 of the vehicle. The outputting of the created motion sickness reduction information may include outputting of the gradation change created by the side window 1630 or the sunroof 1640.

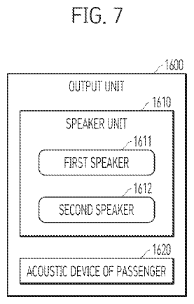

FIG. 7 is a diagram for explaining the output unit 1600 of FIG. 1 in more detail.

In the motion sickness reduction system 1000 according to an embodiment of the present disclosure, the output unit 1600 may output the motion sickness reduction information created through the processor 1500. In more detail, referring to FIG. 7, the output unit 1600 may include a speaker unit 1610 and an acoustic device 1620 of the passenger. Here, the speaker unit 1610 may include a first speaker 1611 located at one side of a seat headrest 10 located in the vehicle and a second speaker 1612 located at the other side.

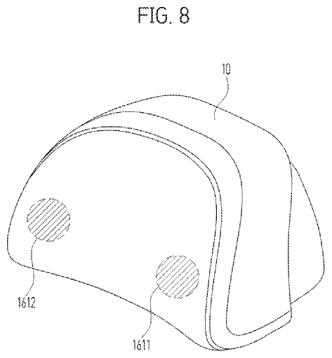

FIG. 8 is a diagram for explaining a spatial acoustic sound that reflects the real-time driving information of the vehicle output through the seat headrest 10 in the motion sickness reduction system 1000 according to an embodiment of the present disclosure.

Referring to FIGS. 1 and 7, in the motion sickness reduction system 1000 according to an embodiment of the present disclosure, the output unit 1600 may output the motion sickness reduction information created by the processor 1500 through the speaker unit 1610 of the seat headrest 10 located in the vehicle. Here, the motion sickness reduction information created by the processor 1500 may include a spatial acoustic sound that reflects the real-time driving information of the vehicle. In addition, the spatial acoustic sound created through the processor 1500 may be sequentially output from the first speaker 1611 to the second speaker 1612 depending on a rotation direction of the vehicle.

FIG. 8 is a diagram for explaining a spatial acoustic sound that reflects the real-time driving information of the vehicle output through the seat headrest 10 in the motion sickness reduction system 1000 according to an embodiment of the present disclosure, and the first speaker 1611 may be built at a left side of the seat headrest 10, and the second speaker 1612 may be built at a right side of the seat headrest 10. That is, two speakers may be built at the left and right sides of the seat headrest 10 to output the above-described spatial acoustic sound.

For example, when the vehicle rotates, sound may be sequentially output from the first speaker 1611 to the second speaker 1612, or from the second speaker 1612 to the first speaker 1611, and thus the passenger may auditorily recognize driving information according to a rotation direction of the vehicle.

Thus, a difference between a motion actually felt by the passenger in relation to real-time driving information of the vehicle and a motion perceived by the brain of the passenger may be reduced, thereby reducing motion sickness of the passenger.

Referring to FIGS. 1 and 7, in the motion sickness reduction system 1000 according to an embodiment of the present disclosure, the processor 1500 may generate spatial acoustic sounds with different tones, intervals, and cycles based on the real-time driving information of the vehicle.

For example, the tone of the spatial acoustic sound that is output according to the speed of the vehicle may be different. Alternatively, the cycle of the spatial acoustic sound that is output according to a rotation radius of the vehicle may vary. In other words, a type of spatial acoustic sound that is output according to the speed, acceleration, and driving direction of the vehicle may vary by reflecting the real-time driving information of the vehicle.

In addition, the motion sickness reduction system 1000 according to an embodiment of the present disclosure may output the spatial acoustic sound based on input of the passenger, received through the aforementioned user input unit 1200.

Therefore, the motion sickness reduction system 1000 according to an embodiment of the present disclosure may generate the spatial acoustic sound providing various types of sounds and the passenger may also choose and output a favorite spatial acoustic sound through the user input unit 1200.

Referring back to FIGS. 1 and 7, in the motion sickness reduction system 1000 according to an embodiment of the present disclosure, the output unit 1600 may output the spatial acoustic sound reflecting the real-time driving information of the vehicle through an acoustic device 1620 of the passenger connected to the vehicle.

That is, the aforementioned acoustic device 1620 may be output to the acoustic device 1620 of the passenger connected wirelessly or by wire through the communication unit 1300, and thus the passenger may auditorily recognize the real-time driving information of the vehicle.

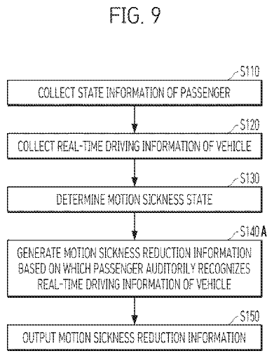

FIG. 9 is a diagram for explaining a method of reducing motion sickness according to an embodiment of the present disclosure.

First, state information of a passenger may be reduced (S110), and the real-time driving information of the vehicle may be collected from a sensor located in the vehicle (S120). Then, the motion sickness state of the passenger may be determined based on the collected state information (S130), and the motion sickness reduction information based on which the passenger auditorily recognizes the real-time driving information of the vehicle may be created based on the determined motion sickness state (S140A). Motion sickness of the vehicle passenger may be reduced by outputting the created motion sickness reduction information (S150).

In more detail, as described above with reference to FIGS. 1, 7, and 8, the creating of the motion sickness reduction information in the method of reducing motion sickness according to an embodiment of the present disclosure may include creating the spatial acoustic sound reflecting the real-time driving information of the vehicle. The outputting of the created motion sickness reduction information may include sequentially outputting sound from the first speaker 1611 to the second speaker 1612 according to a rotation direction of the vehicle.

Here, the creation of the spatial acoustic sound may include creating of the spatial acoustic sound with different tones, intervals, and cycles based on the real-time driving information of the vehicle.

The outputting of the motion sickness reduction information generated in the motion sickness reduction method according to an embodiment of the present disclosure may include outputting a spatial acoustic sound that reflects real-time driving information of the vehicle through the acoustic device 1620 of the passenger connected to the vehicle.

In summary, the motion sickness reduction system and method according to the present disclosure may output motion sickness reduction information based on which the passenger visually recognizes the real-time driving information of the vehicle, and thus reduce a difference between a motion actually felt by the passenger and a motion perceived by the brain of the passenger to reduce motion sickness of the vehicle passenger. In addition, motion sickness of the vehicle passenger may be reduced by outputting various visual contents to reduce motion sickness to the vehicle passenger through a display or window located in the vehicle.

The motion sickness reduction system and method according to the present disclosure may output motion sickness reduction information based on which the passenger visually recognizes the real-time driving information of the vehicle, and thus reduce a difference between a motion actually felt by the passenger and a motion perceived by the brain of the passenger to reduce motion sickness of the vehicle passenger.

Motion sickness of the vehicle passenger may be reduced by outputting various visual contents to reduce motion sickness to the vehicle passenger through a display or window located in the vehicle.

In addition, motion sickness of the vehicle passenger may be reduced by outputting various auditory contents for reducing motion sickness to the vehicle passenger through a seat headrest or a passenger audio device located in the vehicle.

It will be appreciated by persons skilled in the art that the effects that could be achieved with the disclosure are not limited to what has been particularly described hereinabove and other advantages of the present will be more clearly understood from the above detailed description.

The above detailed description is to be construed in all aspects as illustrative and not restrictive. The scope of the present disclosure should be determined by reasonable interpretation of the appended claims and all changes coming within the equivalency range of the present disclosure are intended to be embraced in the scope of the present disclosure.

What is claimed is:

1. A motion sickness reduction system of a passenger of a vehicle, the motion sickness reduction system comprising:
a state information acquirer configured to collect state information of the passenger;
a behavior information acquirer configured to collect real-time driving information of the vehicle from a sensor located in the vehicle;
a processor configured to:
determine a motion sickness state of the passenger based on the collected state information; and
generate motion sickness reduction information based on the determined motion sickness state; and
an output unit configured to output the generated motion sickness reduction information,
wherein the generated motion sickness reduction information output by the output unit is at least one of visually recognizable by the passenger, auditorily recognizable by the passenger, or a combination thereof.

2. The motion sickness reduction system of claim 1, wherein the output unit is further configured to output information through at least one of a display, a front window located in the vehicle, or a combination thereof.

3. The motion sickness reduction system of claim 2,
wherein the motion sickness reduction information generated by the processor includes an augmented reality video reflecting the real-time driving information of the vehicle, and
wherein the generated augmented reality video is output to at least one of the display, a lower end of the front window, or a combination thereof.

4. The motion sickness reduction system of claim 2, wherein the display comprises:
a front display located on a front side of the vehicle; and
a rear seat entertainment (RSE) display located on a roof of the vehicle.

5. The motion sickness reduction system of claim 4, further comprising a driving module located on the roof of the vehicle,
wherein the driving module is configured to rotate or position the RSE display.

6. The motion sickness reduction system of claim 1, wherein the output unit is further configured to output information through at least one of a side window, a sunroof located in the vehicle, or a combination thereof.

7. The motion sickness reduction system of claim 6, wherein the motion sickness reduction information generated by the processor includes a gradation change reflecting the real-time driving information of the vehicle through at least one of a polymer-dispersed liquid crystal (PDLC) film located in the side window, the sunroof, or a combination thereof.

8. The motion sickness reduction system of claim 1, wherein the generated motion sickness reduction information that is recognized by the passenger is comparable with actual motion sickness felt by the passenger.

9. The motion sickness reduction system of claim 1,
wherein the output unit is further configured to output information through a speaker unit of a seat headrest located in the vehicle; and
wherein the speaker unit includes a first speaker located at one side of the seat headrest and a second speaker located at another side of the seat headrest.

10. The motion sickness reduction system of claim 9,
wherein the motion sickness reduction information generated by the processor includes a spatial acoustic sound that reflects the real-time driving information of the vehicle; and
wherein the generated spatial acoustic sound is sequentially output from the first speaker to the second speaker depending on a rotation direction of the vehicle.

11. The motion sickness reduction system of claim 10, wherein the processor is further configured to generate the spatial acoustic sounds with different tones, intervals, and cycles based on the real-time driving information of the vehicle.

12. The motion sickness reduction system of claim 11, further comprising:
a user input unit configured to receive output of the passenger,
wherein the output unit is further configured to output the spatial acoustic sound based on the received output of the passenger.

13. The motion sickness reduction system of claim 1, wherein the output unit is further configured to output a spatial acoustic sound reflecting the real-time driving information of the vehicle through an acoustic device of the vehicle.

14. A method of reducing motion sickness of a passenger of a vehicle, the method comprising:
collecting state information of the passenger;
collecting real-time driving information of the vehicle from a sensor located in the vehicle;
determining a motion sickness state of the passenger based on the collected state information;
generating motion sickness reduction information based on the determined motion sickness state; and
outputting the generated motion sickness reduction information,
wherein the generated motion sickness reduction information that is output is at least one of visually recognizable by the passenger, auditorily recognizable by the passenger, or a combination thereof.

15. The method of claim 14,
wherein the generating of the motion sickness reduction information includes generating an augmented reality video reflecting the real-time driving information of the vehicle; and
wherein the outputting of the generated motion sickness reduction information includes outputting the generated augmented reality video on at least one of a display, a lower end of a front window located in the vehicle, or a combination thereof.

16. The method of claim 14,
wherein the generating of the motion sickness reduction information includes changing a gradation reflecting the real-time driving information of the vehicle through a polymer-dispersed liquid crystal (PDLC) film located in at least one of a side window, a sunroof of the vehicle, or a combination thereof; and
wherein the outputting of the generated motion sickness reduction information includes outputting of the generated gradation change on at least one of the side window, the sunroof, or a combination thereof.

17. The method of claim 14, wherein the generated motion sickness reduction information that is recognized by the passenger is comparable with actual motion sickness felt by the passenger.

18. The method of claim 14,
wherein the generating of the motion sickness reduction information includes generating a spatial acoustic sound that reflects the real-time driving information of the vehicle; and
wherein the outputting of the generated motion sickness reduction information includes outputting information from a first speaker to a second speaker depending on a rotation direction of the vehicle.

19. The method of claim 18, wherein the generating of the spatial acoustic sound includes generating the spatial acoustic sounds with different tones, intervals, and cycles based on the real-time driving information of the vehicle.

20. The method of claim 14, wherein the outputting of the generated motion sickness reduction information includes outputting a spatial acoustic sound that reflects the real-time driving information of the vehicle through an acoustic device of the passenger connected to the vehicle.
